(19) United States
(12) Patent Application Publication
(10) Pub. No.: US 2008/0075680 A1
(43) Pub. Date: Mar. 27, 2008

(54) Culture of microorganisms endogenous to plants and products thereof

(76) Inventor: Jonathan Y. Chen, Gwynn Oak, MD (US)

Correspondence Address: LITMAN LAW OFFICES, LTD., P.O. BOX 15035, CRYSTAL CITY STATION, ARLINGTON, VA, 22215, US

(21) Appl. No.: 11/898,512

(22) Filed: Sep. 12, 2007

Related U.S. Application Data
(60) Provisional application No. 60/847,362, filed on Sep. 27, 2006.

(51) Int. Cl. A61K 8/00; A61P 43/00; C12N 1/02

(52) U.S. Cl. 424/70.1; 435/261

(57) ABSTRACT

The culture of microorganisms endogenous to plants is a process for increasing the number of such microorganisms and collecting the enriched culture media for nutritional, pharmaceutical and cosmetic use. The process includes the steps of: (a) separating plant components (e.g., leaves, fruit, etc.) into smaller parts; (b) immersing the smaller plant parts in a culture medium of an aqueous solution of sodium chloride and sucrose; (c) permitting the immersed parts to stand in the culture medium for between about 7-14 days; (d) removing the smaller plant parts from the culture medium; (e) dividing the culture medium into two portions; (f) diluting each of the portions 1:2 with water; (g) permitting the diluted portions to stand between about 7-14 days to ferment; (h) filtering the diluted portions; and (i) collecting the supernatant. Steps (e) through (g) may be repeated as often as desired. The process may also include maintaining the concentration of sucrose.

### CROSS-REFERENCE TO RELATED APPLICATION

This application claims the benefit of U.S. Provisional Patent Application Ser. No. 60/847,362, filed Sep. 27, 2006.

### BACKGROUND OF THE INVENTION

1. Field of the Invention

The present invention relates to methods for culturing microorganisms, and particularly to the culture of microorganisms endogenous to plants and products thereof that are useful for nutritional, pharmaceutical, and cosmetic use.

2. Description of the Related Art

Many substances and compositions found to be beneficial or useful for nutritional, pharmaceutical, or cosmetic purposes are derived from plants. Historically it has generally been assumed that the active ingredient in such substances and compositions are compounds resulting exclusively from plant metabolism, i.e., from photosynthesis and as by-products from anabolic and catabolic processes catalyzed by enzymes produced in accordance with the plant's DNA and RNA.

Recently, however, it has been shown that, at least in some cases, the nutritional, pharmaceutical, or cosmetic properties that make these substances and compositions valuable result from the secretions of microorganisms endogenous to, or hosted by, the plant, the secretions being effective either by themselves or in combination with the natural juices, fluids, tissues, or metabolic by-products of plant metabolism. For example, U.S. Pat. Nos. 6,290,964 and 6,551,631, issued to Shupe et al. on Sep. 18, 2001 and Apr. 22, 2003, respectively, appear to attribute the antimicrobial activity of the Aloe vera gel described by Coats in U.S. Pat. No. 5,356,811, at least in part, to ten species of bacteria found in the leaves of the aloe plant.

While gels, extracts, powders, and other products derived from such diverse plants and herbs as Aloe vera, pomegranate, kukui, mangosteen, ginseng root, cactus (including prickly pear cactus), passion fruits, etc. have been found useful, at least to some degree, for nutritional, pharmaceutical and cosmetic purposes, it would be desirable to concentrate the active ingredients derived from these plants and the microorganisms residing therein to produce a more potent effect, as well as for the development of new uses for the plant derivatives.

Thus, the culture of microorganisms endogenous to plants and the products thereof solving the aforementioned problems is desired.

### SUMMARY OF THE INVENTION

The culture of microorganisms endogenous to plants is a process for increasing the number of such microorganisms and collecting the enriched culture media for nutritional, pharmaceutical and cosmetic use. The process includes the steps of: (a) separating plant components (e.g., leaves, fruit, etc.) into smaller parts; (b) immersing the smaller plant parts in a culture medium of an aqueous solution of sodium chloride and sucrose; (c) permitting the immersed parts to stand in the culture medium for between about 7-14 days; (d) removing the smaller plant parts from the culture medium; (e) dividing the culture medium into two portions; (f) diluting each of the portions 1:2 with water; (g) permitting the diluted portions to stand to ferment for between about 7-14 days; (h) filtering the diluted portions; and (i) collecting the supernatant. Steps (e) through (g) may be repeated as often as desired. The process may also include maintaining the concentration of sucrose.

These and other features of the present invention will become readily apparent upon further review of the following specification.

### DETAILED DESCRIPTION OF THE PREFERRED EMBODIMENTS

The present invention relates to the culture of microorganisms endogenous to plants and the products thereof, which have nutritional, pharmaceutical, and cosmetic uses. Exemplary plants that the process can be applied to include Aloe vera, pomegranate, kukui, mangosteen, ginseng root, cactus (including prickly pear cactus), and passion fruits, although these plants have been selected for purposes of illustration of the method, which may be applied more broadly to any plant having endogenous microorganisms residing therein.

Stated broadly, the culture of microorganisms endogenous to plants is a process that includes the steps of: (a) separating plant components (e.g., leaves, fruit, etc.) into smaller parts; (b) immersing the smaller plant parts in a culture medium of an aqueous solution of sodium chloride and sucrose; (c) permitting the immersed parts to stand in the culture medium for between about 7-14 days; (d) removing the smaller plant parts from the culture medium; (e) dividing the culture medium into two portions; (f) diluting each of the portions 1:2 with water; (g) permitting the diluted portions to stand to ferment for between about 7-14 days; (h) filtering the diluted portions; and (i) collecting the supernatant. Steps (e) through (g) may be repeated as often as desired. The process may also include maintaining the concentration of sucrose. The process will now be illustrated by describing its application to several plants.

Aloe vera is a member of the aloe genus, and is often called aloe barbadensis. Aloe vera is a plant with yellow flowers and tough, spearlike leaves that grow up to twenty inches long and five inches across the base. The edges of the leaf have a sawtooth configuration. The leaf has three layers, including a tough outer skin, a corrugated layer just below the skin, and a pulp or inner layer of cells containing vacuoles of a semisolid, gelatinous transparent gel. The corrugated layer contains a yellow latex juice.

Various products are derived from Aloe vera, including Aloe vera gel, which is a naturally occurring gel obtained by stripping away the outer layer of the leaf; Aloe vera concentrate, which is obtained by removing water from the gel; Aloe vera juice, which is a liquid that can be taken internally and is at least one-half Aloe vera gel; and Aloe vera latex, which is a bitter yellow liquid derived from the rind of the leaves and is often used as a cathartic or laxative. Aloe vera is known to contain many compounds exhibiting biological activity, including anthraquinones, polysaccharides, prostaglandins, vitamins, and minerals. Aloe vera is reputed to have antimicrobial activity, antifungal activity, antiviral activity (particularly relating to the AIDS virus), immune system effects, antiallergy effects, antiinflammatory effects, as well as promoting tissue formation in wound healing, serving as a tonic for peptic ulcers and other gastrointestinal problems, and having moisturizing and emollient effects for cosmetic applications.

In the present invention, the leaves are cleaned with cold tap water containing about 0.01% household detergent, rinsed with cold water, and then rinsed with distilled water containing 0.8-1.3M sodium chloride. The inner gel and inside tissues are removed from the leaves. In particular, razor-sharp knives are used to dissect out the outside skin (the epidermis or rind). The cortex, including the chloroplasts, the sap (vascular bundles with xylem and phloem), and the pith (including part of the vascular bundles, lacunars parenchyma or mesophyll, the liquid layer known as mucilage, and the spongy parenchyma) is completely scraped off from the rind twice, once parallel to the leaf and another time perpendicular to the leaf. The rind is rinsed of all gel and mucilage with distilled water, and finally salt water. This process should remove all of the aloin-containing sap and mucilage from the rind, which is then ready to be cut for culturing. The sheaths of the leaves or stems are cut into roughly 2-inch cubes (only the rind is used) and dropped into a transparent container containing a solution that is 0.1-0.75M food grade NaCl and 0.1-0.40M food grade sucrose. The container is stoppered or capped, and the solution is left standing at room temperature (about 20-25° C., more preferably, about 72-75° F. or 22-24° C.) for 7-14 days.

The leaf portions are then removed from the solution, which is then divided equally between two containers and diluted 1:2 with distilled water. The culture in both containers is then made between 0.05-0.2 M sucrose. Both containers are left standing at room temperature for another 7-14 days. The latter division and dilution may be repeated as many times as desired. After 2-3 serial dilutions and incubation periods, a thick white-brown layer of microorganisms can be observed at the bottom of the bottle. After the desired number of dilutions, the supernatant is then allowed to stand at room temperature to ferment for at least two weeks and filtered through a 0.45 micron filter before use. The container is stoppered or capped at all times, except when splitting and diluting the solution, and except to release gas build up in the container (a few minutes every three-four days).

The supernatant from the Aloe vera cultures has been found to regenerate hair in at least six subjects when applied topically. The hair regenerates through a gradient path, the hair starting to regenerate where the hair was lost last. The regenerated hair growth spread all over the head. The process generally takes six months to cover the whole head with hair, but some people regenerated hair more slowly.

The supernatant has also been found to relieve pain and regenerate cartilage in arthritic joints in various subjects. The supernatant is also useful for the relief of muscular pain, cancer pain (epithelioid hemangiothelioma) and the pain and itching incident to sunburn and insect bites. For these applications, the supernatant is sprayed on the affected area at least twice daily. Pain in the teeth and gums may be relieved by rinsing with one tablespoon of the supernatant. Finally, stomachache has been relieved and the loss of menstrual periods has been rectified by ingestion of the supernatant.

When used for these purposes, the Aloe vera culture is not harvested until one-two months after the last splitting of the media. This may be a fermentation period in which the culture is converted into an alcohol form, since the composition emits an aldehyde odor when sprayed on the affected area, which may represent oxidation of the alcohol to form an aldehyde.

The pomegranate (Punica granatum) is a fruit having its origins in the Middle East and Asia, but which is now grown on many continents. Pomegranates are produced by the pomegranate tree, and are a round fruit having a hard red and yellow outer skin and a semi-sweet pulp with numerous seeds enclosed in a membrane. The fruit is quite juicy, and the juice and pericarp (peel) contain many polyphenols, including flavonoids, anthocyanins, and tannins.

In the present invention, pomegranate fruits are cleaned with cold tap water containing 0.001% household detergent, rinsed in cold water, and then rinsed with distilled water 0.4-1.0M in NaCl. The fruits are cut into roughly ½″ cubes and placed in a transparent container containing a solution of 0.075-0.2M food grade NaCl and 0.05-0.2M food grade sucrose. The solution is allowed to stand at room temperature (about 20-25° C., more preferably, about 72-75° F. or 22-24° C.) for 7-14 days. The pomegranate pieces are then removed from the solution, which is divided equally between two containers, diluted 1:2, and made 0.05-0.2M sucrose.

The cultures are allowed to stand another 7-14 days to ferment at room temperature. The latter division and dilution may be repeated as many times as desired. After the desired number of dilutions, the supernatant is then allowed to stand at room temperature for at least two weeks and filtered through a 0.45 micron before use.

The supernatant from the pomegranate cultures has been found to lower blood pressure and to sedate the body through stimulation of the Vagus Nerve Terminal in the stomach. The pomegranate supernatant has also been found to relieve the nausea that sometimes occurs as a side effect from certain prescription medications.

Kukui fruit comes from the kukui or candlenut tree (Aleurites molucanna), which is native to Polynesia and Hawaii. The fruit is called a drupe, and may be a round fruit containing a single seed, or ellipsoidal containing two seeds. The fruit has a green outer layer (exocarp), a hard or crusty inner shell (the stone or endocarp), and a seed that is rich in oil contained within the endocarp. The raw fruit is a cathartic and is laxative when roasted.

According to the present invention, kukui fruits are cleaned with cold tap water containing 0.001% household detergent, rinsed in cold water, and then rinsed with distilled water 0.4-1.0M in NaCl. The fruits, rid of the seeds, are cut into roughly ½″ cubes and placed in a transparent container containing a solution of 0.075-0.2M food grade NaCl and 0.05-0.2M food grade sucrose. The solution is allowed to stand at room temperature (about 20-25° C., more preferably, about 72-75° F. or 22-24° C.) for 7-14 days. The kukui pieces are then removed from the solution, which is divided equally between two containers, diluted 1:2, and made 0.05-0.2M sucrose.

The cultures are allowed to stand another 7-14 days at room temperature to ferment. The latter division and dilution may be repeated as many times as desired. After the desired number of dilutions, the supernatant is then allowed to stand at room temperature for at least two weeks and filtered through a 0.45 micron before use.

The supernatant from the kukui cultures has been found to be useful for cosmetic purposes in removing blemishes and acne from facial tissues.

Mangosteen is a tropical fruit produced by the mangosteen tree (Garcinia mangostana L.) in Southeast Asia, Central Africa, and other tropical climates. The fruit is round and dark purple or reddish purple in color. The rind is ¼″-⅜″ thick and contains a bitter yellow latex, as well as purple juice. In the center of the fruit are 4-8 edible white pods, which are the seed arils. Mangosteen fruit contains xanthones, and is believed to be an antioxidant having antibacterial, antiviral, and antifungal properties.

In the present invention, mangosteen fruits are cleaned with cold tap water containing 0.001% household detergent, rinsed in cold water, and then rinsed with distilled water 0.4-1.0M in NaCl. The fruits, rid of the seeds, are cut into roughly ½″ cubes and placed in a transparent container containing a solution of 0.075-0.2M food grade NaCl and 0.05-0.2M food grade sucrose. The solution is allowed to stand at room temperature (about 20-25° C., more preferably, about 72-75° F. or 22-24° C.) for 7-14 days. The mangosteen pieces are then removed from the solution, which is divided equally between two containers, diluted 1:2, and made 0.05-0.2M sucrose.

The cultures are allowed to stand another 7-14 days at room temperature. The latter division and dilution may be repeated as many times as desired. After the desired number of dilutions, the supernatant is then allowed to stand at room temperature to ferment for at least two weeks and filtered through a 0.45 micron before use.

The supernatant from the mangosteen cultures has been found to be useful as a nutritional supplement for its antioxidant properties.

Ginseng root is generally produced from Panax ginseng, which is native to China and Korea, or its American cousin, Panax quinquefolium. Ginseng is an herbaceous plant with a taproot and produces flowers in an umbel. Roots from the wild plant are highly prized, but most commercially available ginseng is derived from the roots of cultivated plants ranging from three to six years old. The root is usually about 3-4 millimeters in diameter and about 10 centimeters long.

Ginseng has long been used as a general tonic, and has been used in folk medicine to treat a wide variety of disease and pathological conditions. Ginseng contains about thirteen triterpenoid saponins collectively referred to as ginsenosides, and several other compounds having pharmaceutical value, including panacene and other peptides, steroids, polyacetylene derivatives, carbohydrates, polysaccharides, vitamins, amino acids, minerals, and flavonoids.

According to the present invention, microorganisms residing in the roots are cultured. The roots are cleaned with cold tap water containing 0.001% household detergent, rinsed in cold water, and then rinsed with distilled water 0.4-1.0M in NaCl. The roots are cut into roughly ½″ cubes and placed in a transparent container containing a solution of 0.075-0.2M food grade NaCl and 0.05-0.2M food grade sucrose. The solution is allowed to stand at room temperature (about 20-25° C., more preferably, about 72-75 ° F. or 22-24° C.) for 7-14 days. The root pieces are then removed from the solution, which is divided equally between two containers, diluted 1:2, and made 0.05-0.2M sucrose.

The cultures are allowed to stand another 7-14 days at room temperature. The latter division and dilution may be repeated as many times as desired. After the desired number of dilutions, the supernatant is then allowed to stand at room temperature to ferment for at least two weeks and filtered through a 0.45 micron before use.

Cactus, and particularly the prickly pear cactus (various species of Opuntia), has been used for medicinal purposes by Native Americans and Mexicans. The stems (the fleshy pads or cladodes) were cleaned of their spines, split in half, warmed, and used to treat rheumatism, asthma, earaches, etc., and the pads were also used as poultices for insect bite, snakebite, burns, rashes, sunburn, and minor abrasions.

In the present invention, the pads are cleaned with cold tap water containing 0.001% household detergent, rinsed in cold water, and then rinsed with distilled water 0.4-1.0M in NaCl. The inner gel or inside tissues are removed from the pads. The sheaths of the leaves or stems are cut into roughly 2″ cubes and placed in a transparent container containing a solution of 0.075-0.2M food grade NaCl and 0.05-0.2M food grade sucrose. The solution is allowed to stand at room temperature (about 20-25° C., more preferably, about 72-75° F. or 22-24° C.) for 7-14 days. The cactus pieces are then removed from the solution, which is divided equally between two containers, diluted 1:2, and made 0.05-0.2M sucrose.

The cultures are allowed to stand another 7-14 days at room temperature. The latter division and dilution may be repeated as many times as desired. After the desired number of dilutions, the supernatant is then allowed to stand at room temperature for at least two weeks to ferment and filtered through a 0.45 micron before use.

Passion fruit, Passiflora edulis or Passiflora incarnate is a tropical fruit native to North and South America, but now cultivated in many tropical areas of the world. The various species of Passiflora produce fruits that are either a yellow fruit about the size of a grapefruit, or a smaller purple fruit. The fruit has historically been used by Native Americans to treat insomnia, as an antidepressant, and for its antispasmodic and analgesic properties. The plant contains various alkaloids, including beta-carbolene harmala compounds that are monoamine oxidase inhibitors, various flavonoids, sterols, and gums.

In the present invention, passion fruits are cleaned with cold tap water containing 0.001% household detergent, rinsed in cold water, and then rinsed with distilled water 0.4-1.0M in NaCl. The fruits, rid of the seeds, are cut into roughly 2″ cubes and placed in a transparent container containing a solution of 0.075-0.2M food grade NaCl and 0.05-0.2M food grade sucrose. The solution is allowed to stand at room temperature (about 20-25° C., more preferably, about 72-75° F. or 22-24° C.) for 7-14 days. The passion fruit pieces are then removed from the solution, which is divided equally between two containers, diluted 1:2, and made 0.05-0.2M sucrose.

The cultures are allowed to stand another 7-14 days at room temperature. The latter division and dilution may be repeated as many-times as desired. After the desired number of dilutions, the supernatant is then allowed to stand at room temperature to ferment for at least two weeks and filtered through a 0.45 micron before use.

It is to be understood that the present invention is not limited to the embodiments described above, but encompasses any and all embodiments within the scope of the following claims.

I claim:

1. A method for culture of microorganisms endogenous to plants, comprising the steps of:
(a) separating plant components into smaller parts;
(b) immersing the smaller plant parts in a culture medium comprising an aqueous solution of sodium chloride and sucrose;
(c) permitting the immersed parts to stand in the culture medium for between about 7-14 days;
(d) removing the smaller plant parts from the culture medium;
(e) dividing the culture medium into two portions;
(f) diluting each of the portions 1:2 with water;
(g) permitting the diluted portions to stand for between about 7-14 days to ferment;
(h) filtering the diluted portions; and
(i) collecting the supernatant.

2. The method for culture of microorganisms according to claim 1, further comprising the steps of:
(j) after step (g) and before step (h), diluting each of the 1:2 diluted portions an additional 1:2 with water to form 1:4 diluted portions; and
(k) after step (0) and before step (h), permitting the 1:4 diluted portions to stand and ferment for at least seven days.

3. The method for culture of microorganisms according to claim 1, wherein the plant components are selected from the group consisting of Aloe vera leaf rinds with leaf cortex removed, pomegranate fruits, kukui fruits, mangosteen fruits, ginseng roots, cactus pads, and passion fruits.

4. A composition, comprising the supernatant collected according to the method of claim 3.

5. The method for culture of microorganisms according to claim 1, wherein the plant components comprise Aloe vera leaves, the method further comprising the steps of:
dissecting out rind from the Aloe vera leaves;
scraping cortex from the rind; and
rinsing the scraped rind to remove aloin-containing sap and mucilage.

6. The method for culture of microorganisms according to claim 5, wherein step (a) comprises cutting the scraped and rinsed rind in cubes of about two inches.

7. The method for culture of microorganisms according to claim 6, wherein said culture medium is between about 0.1-0.75M food grade sodium chloride and 0.1-0.40M food grade sucrose.

8. A method for regenerating lost hair, comprising the step of administering an effective amount of the supernatant collected from the culture of microorganisms according to the method of claim 7 to a subject in need of hair restoration.
